(19) United States
(12) Patent Application Publication
(10) Pub. No.: US 2010/0298623 A1
(43) Pub. Date: Nov. 25, 2010

(54) INTRA-SESSION CONTROL OF TRANSCRANIAL MAGNETIC STIMULATION

(76) Inventors: David J. Mishelevich, Playa del Rey, CA (US); M. Bret Schneider, Portola Valley, CA (US)

Correspondence Address: SHAY GLENN LLP, 2755 CAMPUS DRIVE, SUITE 210, SAN MATEO, CA, 94403, US

(21) Appl. No.: 12/680,749

(22) PCT Filed: Oct. 24, 2008
(86) PCT No.: PCT/US08/81048

§ 371 Date: Jul. 14, 2010

(30) Foreign Application Priority Data
Oct. 24, 2007 (US) 60982141

(51) Int. Cl. A61N 2/04

(52) U.S. Cl. 600/13

(57) ABSTRACT

Described herein are methods for controlling Transcranial Magnetic Stimulation during or within a session, where direct immediate patient reported feedback is utilized to assess the effect and optimize the treatment in real time. These methods may be applicable to superficial repetitive Transcranial Magnet Stimulation (rTMS) or deep-brain stereotactic Transcranial Magnetic Stimulation (sTMS). Examples of therapies that may benefit from these methods include TMS treatment of: acute pain (e.g., during dental procedures or bunionectomies), depression, or Parkinson's Disease, to name only a few. TMS systems and devices including or more patient inputs that may be used to perform these methods are also described.

### CROSS REFERENCE TO RELATED APPLICATIONS

This application claims priority to the following application: U.S. Provisional Patent Application Ser. No. 60/982,141, filed on Oct. 24, 2007, titled “INTRA-SESSION CONTROL OF TRANSCRANIAL MAGNETIC STIMULATION.” This application is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD OF THE INVENTION

The devices and methods described herein relate generally to control of moving, positioning, and activating electromagnets generating magnetic fields used for Transcranial Magnetic Stimulation.

### BACKGROUND OF THE INVENTION

Transcranial Magnetic Stimulation (TMS) of targets within the brain or other parts of the nervous system may modulate neural activity, and may be used to treat a variety of disorders, behaviors and indications. For example, positive outcomes for treatment of depression refractory to drug treatment have been demonstrated with rTMS (repetitive Transcranial Magnetic Stimulation, Avery et al., 2005). rTMS is believed to work indirectly because the superficial stimulation of the dorsolateral pre-frontal cortex is carried by nerve fibers to the deeper cingulate gyrus. TMS of both superficial and deep-brain regions may also be used to treat other disorders or conditions, such as acute or chronic pain, addiction, obesity, and obsessive compulsive disorder (OCD).

TMS therapies for many of these disorders is likely to be successful only if stimulation of deep-brain target regions can be achieved. Deep brain targets are of particular interest for TMS, but practical deep-brain TMS has been difficult to achieved, because stimulation at depth must be performed without over-stimulating superficial tissues. Recently, Schneider and Mishelevich, U.S. patent application Ser. No. 10/821,807 and Mishelevich and Schneider, U.S. patent application Ser. No. 11/429,504, have described methods for achieving TMS stimulation of deep brain regions without over stimulating (or in some cases even stimulating) regions superficial to the deep brain region.

In operation, TMS may be performed on a patient by a medical professional that operates the system. The medical professional adjusts the system, e.g., the magnet(s), to target one or more brain regions, and will typically use brain scans or maps of the particular patent's brain (e.g., using MRI, or other imaging techniques). In addition to positioning the magnet(s), the practitioner may also set the intensity (e.g., power) and frequency of firing of the TMS electromagnets. Thus, the targeting and stimulation levels are usually done in an open-loop system, without substantial functional feedback from the patient or patient being treated. This type of control does not allow functional feedback, and may be less accurate and also less effective than a system that would somehow directly confirm adequate stimulation of the appropriate brain region necessary for achieving therapeutic effect. Described herein are systems and methods that allow direct patient feedback based on acute effects of TMS that have are correlated with the target therapy.

In contrast with the direct patient feedback described herein, simple indirect patient feedback is known. For example, indirect physiologic feedback during a TMS session includes brain imaging during the course of a TMS procedure. For example, EEG and fMRI instrumentation have been employed. Use of the EEG domain is described in Ives and Pascual-Leone, U.S. Pat. Nos. 6,266,256 and 6,571,123. Use of the fMRI domain are described in Ives, et al. U.S. Pat. No. 6,198, 958 and Bohning and George, U.S. patent application Ser. No. 10/991,129. These employ interleaving of TMS and fMRI. George et al., U.S. patent application Ser. No. 10/521,373 describe using TMS to prevent a patient from deceiving the user, but not for alleviating a condition; the process is also used in conjunction with fMRI.

Motor feedback based on stimulation parameters has also been described (Riehl, U.S. Pat. No. 7,104,947), but is not applicable to the conditions addressed herein. Fox and Lancaster, U.S. Pat. No. 7,087,008 teach a robotic system for positioning TMS coils involving PET scanning to locate the target, but the system does not use direct patient-reported feedback. Tanner (U.S. Pat. Nos. 6,830,544 and 7,239,910) has described presentation of stimuli (e.g., optical, auditory, or olfactory) in the usual way and then adjusting applied TMS to reproduce the same sensations as closely as possible, in some cases using passive markers for navigation. Tanner et al., in U.S. Pat. No. 7,008,370, describe matching coordinates of a simulation model generated from MRI data with a model of the TMS induction device, positioning the electromagnet on the head, and stimulating using the electromagnet to get a response (such as an EMG response in the forearm of the patient) indicating the mapping. These mapping methods are used as part of a mapping method for investigating only normal functions and do not deal with treatment.

While the above-described approaches can be useful, they are not applicable in ambulatory settings where the vast numbers of patients will be treated. Further, all of these methods operate by inference, based on generalization of treatment of brain regions, and assume that the desired therapeutic effect will be follow TMS of the patient's brain region, rather than assess the effects of the TMS directly. What is needed is a mechanism to obtain direct immediate patient-reported feedback from the patient and make intra-session adjustments accordingly.

Although patient-reported feedback has been applied with some success in other treatment types, such as implantable electrode stimulation, it has not been applied to TMS therapies. For example, Mayberg and Lazano have previously documented patient-reported immediate feedback in deep brain stimulation using implanted electrodes. They describe patients who reported immediate lifting of depressive systems while undergoing deep brain stimulation using electrodes inserted in the brain tissue.

The methods, devices and systems for TMS described herein all include patient feedback and/or control of the TMS stimulation in a manner that may allow enhanced accuracy and efficacy over previous TMS therapy methods.

### SUMMARY OF THE INVENTION

Described herein are systems and methods for treating a patient with TMS, and particularly deep brain TMS, in which the patient has a least limited control of one or more TMS parameters, such as the position of the TMS magnet(s), the intensity of the TMS stimulation (e.g., applied magnetic field), or the frequency of the TMS stimulation. This patient feedback is based on the patient's acute experience during the TMS stimulation, which may be provoked by a stimulus, or unprovoked.

In general, the patient undergoing the TMS therapy alters one or the TMS parameters (e.g., using one or more inputs) based on one or more acute responses to the TMS procedure. Thus, any of the methods described herein may include feedback from the patient to alter a parameter based on the patient's experience. For example, if the patient is being treated for pain, and the patient does not experience a cessation or lessening of acute pain during TMS treatment, the patient may change the treatment (e.g., move the TMS electromagnet or increase the stimulation intensity or increase the stimulation frequency), until the pain is lessened. In some situations direct and immediate feedback from the patient may be triggered by alleviation of the condition being treated. For example, when treating depression, stimulation with TMS deep within the brain (e.g., using techniques such as Schneider and Mishelevich, U.S. patent application Ser. No. 10/821,807 and Mishelevich and Schneider, U.S. patent application Ser. No. 11/429,504), immediate relief (e.g., positive feelings) may be experienced. Indirect stimulation of the cingulate gyrus by superficial rTMS (repetitive Transcranial Magnetic Stimulation) has already demonstrated immediate increased blood flow with Positive Emission Tomography (PET) using oxygen or glucose-mediated agents.

Acute responses may be triggered or provoked by a stimulus correlated with the disorder, disease or behavior being treated. In some treatments the patient experience tied to the patient's feedback may be related to the disorder being treated. For example, during treatment the patient may experience an immediate symptom reduction (e.g., acute pain, drug addiction). Some conditions to which superficial or deep TMS are applicable may have no immediate acute demonstrable patient-reported effect during the session (e.g., obesity). In such cases, a proxy or surrogate acute response experienced during treatment may be used to trigger patient feedback, and the patient may be instructed or trained to respond to the surrogate. For example, treatment side effects including stimulation site pain, visual disturbances and induced motor activity may be present during the course of a typical treatment session, and one or more of these side effects may be correlated with a desired treatment region. For example, when treating pain or attempting to effect anesthesia/analgesia, the cessation of stimulation site pain may trigger feedback by the patient.

The stimulation applied to any target, including the targets identified herein, may be either up- or down-regulating stimuli. For example, “up regulation” in a particular brain region may mean stimulation at a frequency of about 5 Hz or greater within the target region. Similarly, “down-regulation” of a target region may refer to stimulation at a rate of 1 Hz or less.

In some variations, the acute experience used by the patient to control the TMS therapy may be triggered by a stimulus during (or immediately before) application of the TMS therapy. For example, if treating a disorder such as obsessive compulsive disorder (OCD), the patient may be exposed to a stimulus would normally cause anxiety (e.g., a soiled garment, an unpleasant image, etc.). Other conditions with potential for immediate feedback include addiction and addictive behaviors, in which the patient may be exposed to a stimulus that would normally trigger an emotional response, such as drugs, cigarettes, alcohol or food. This triggering stimulus may be applied during or before TMS treatment, and the patient may then experience an acute reduction in the effect.

For patients having chronic conditions with an acute equivalent (e.g., chronic pain), checking the patient's response to an acute version could permit inferences related to the chronic version to be used for treatment planning. For example, consider a patient with chronic pain. Using our invention either there will be immediate relief or not; in the first case, the given patient will get immediate relief from his or her chronic pain with suitably adjusted TMS. In the second case, If immediate relief from chronic pain does not occur because the chronic pain condition will require repetitive treatments to bring relief, the approach would be to cause the patient to have an acceptable level of acute pain (say by applying a noxious substance such as capsaicin pepper extract) as a surrogate for chronic pain, adjust the TMS parameters to get maximum relief for the acute pain, and use then those same parameters for subsequent TMS treatments of the chronic pain. While the chronic-pain pathway response may not exactly mirror that for acute pain, it would be an excellent place to start.

Although patient feedback/control during the TMS therapy is typically experiential, or based on the patientive experience of the patient, it may also (or alternatively) be controlled by one or more involuntary, unconscious, and/or physiological patient responses. For example, successful TMS treatment may cause an involuntary or physiological response that is not recognized by the patient, such as increase or decrease in heart rate, blood pressure, respiratory rate, etc. This type of ‘involuntary’ patient feedback may also be detected by the system, and may be used to modify the treatment. In some variations, the system may prevent false or erroneous reporting of conscious or volitional feedback by requiring both unconscious and conscious feedback. For example, if treating pain, the system may allow the patient to continue to adjust one or more parameter during TMS treatment (patient control feedback), as long as an ‘unconscious’ patient feedback does not indicate successful treatment (e.g., change in heart rate, blood pressure, etc., indicating alleviation in pain). Alternatively, the unconscious or involuntary patient feedback may be used to select the parameter controlled by the patient or the magnitude of the patient control.

For example, described herein are patient-configurable Transcranial Magnetic Stimulation (TMS) methods that allows a patient to dynamically modify the TMS while a TMS procedure is being performed. In some variations, the methods include the steps of: applying Transcranial Magnetic Stimulation to a first site in the patient's brain, at a first magnetic field intensity and a first stimulation frequency; changing one or more of the site, intensity or the frequency of the TMS stimulation based on input from the patient, wherein the patient changes one or more of the site, intensity or frequency of the TMS stimulation based on the patient's experience of the applied TMS stimulation; and applying Transcranial Magnetic Stimulation to the patient at the new site, intensity or frequency of TMS stimulation.

The method may also include the step of providing a stimulus to prompt a patient experience that is modified during the TMS procedure. Stimulus may be a stimulus that triggers, exacerbates or mimics the disorder, disease or behavior being treated. For example, the trigger may be an image of food when treating obesity/overeating, or a representation (sight/smell) of a drug or alcohol when treating addiction. Thus, the stimulus may comprise a visual stimulus, tactile stimulus, etc.

The step of changing one or more of the site, intensity or the frequency of the TMS stimulation may comprise allowing the patient to manipulate a handheld control to alter one or more of the site, intensity or frequency of the TMS stimulation. For example, the patient may move a joystick, toggle, dial, or other control during treatment. In some variations, the amount of control exerted by the patient during treatment may be limited. As mentioned, it may be limited or gated by unconscious patient feedback or input (e.g., heart rate, etc.). In some variations, the patient control may be limited to control within a range of values. For example, the patient may alter the site, intensity or frequency of the TMS stimulation only within a predetermined range for each of the site, intensity or frequency.

The step of changing one or more of the site, intensity or the frequency of the TMS stimulation may be performed while applying Transcranial Magnetic Stimulation to the patient. In some variations the patient control is exerted between ‘rounds’ of TMS stimulation.

Also described herein are patient-configurable Transcranial Magnetic Stimulation (TMS) methods that allows a patient to dynamically modify the TMS while a TMS procedure is being performed, the method comprising: positioning a plurality of TMS electromagnets to apply electromagnetic energy to a deep brain target site; applying TMS to the target site at a magnetic field intensity and a stimulation frequency; enabling the patient to change one or more of the position of the TMS electromagnet, the intensity of the TMS stimulation, or the frequency of the TMS stimulation based the patient's experience of the applied TMS stimulation; and applying Transcranial Magnetic Stimulation to the patient at the changed position of the TMS electromagnet, intensity of the TMS stimulation, or frequency of TMS stimulation.

As mentioned above, the method may also include providing a stimulus to prompt a patient experience that is modified during the TMS procedure. The stimulus comprises a visual stimulus, a tactile stimulus, a smell, a sound, etc.

As mentioned, the step of enabling the patient to change one or more of the position of the TMS electromagnet, the intensity of the TMS stimulation, or the frequency of the TMS stimulation may include allowing the patient to manipulate a handheld control.

Also described herein are systems for applying Transcranial Magnetic Stimulation (TMS), the systems comprising: at least one TMS electromagnet configured to apply TMS to a site in a patient's brain; a controller configured to control the TMS electromagnet to apply TMS to the site in a patient's brain at a magnetic field intensity and a frequency of stimulation; and a patient feedback input connected to the controller, configured to allow the patient to adjust one or more of the site of application of the TMS in the patient's brain, the magnetic field intensity of the applied TMS, or the frequency of the TMS stimulation during a TMS procedure on the patient.

In any of these systems, a plurality of TMS electromagnets configured to be positioned to apply TMS to a site in a patient's brain at a magnetic field intensity and a frequency of stimulation may be used.

The controller may be configured to coordinate the stimulation applied by a plurality of TMS electromagnets to apply TMS to a deep brain target. The patient feedback input may comprise a joystick, a mouse, a touch screen, a motion sensor, etc. As mentioned, the controller may be configured to limit the adjustment of the site of application of the TMS in the patient's brain, the magnetic field intensity of the applied TMS, and the frequency of the TMS stimulation by the patient feedback input so that these parameters remain within a predetermined range of values.

Also described herein are systems for applying Transcranial Magnetic Stimulation (TMS) including a plurality of TMS electromagnets configured to apply TMS to a deep brain target site in a patient's brain; a controller configured to control the plurality of TMS electromagnets to apply TMS to the target site in the patient's brain at a magnetic field intensity and a frequency of stimulation; and at least one patient feedback input configured to allow the patient to adjust one or more of the site of application of the TMS in the patient's brain, the magnetic field intensity of the applied TMS, or the frequency of the TMS stimulation during a TMS procedure on the patient.

### BRIEF DESCRIPTION OF THE DRAWINGS

FIG. 1 is a flow chart illustrating one variations of the method for intra-session control of TMS.

FIG. 2 shows one variation of a system for patient-configurable TMS.

FIG. 3 is a table of therapies, deep-brain TMS targets and exemplary acute patient feedback.

### DETAILED DESCRIPTION OF THE INVENTION

In general, the devices and methods described herein allow patient feedback based on acute effects during a Transcranial Magnetic Stimulation (TMS) treatment to modify the TMS treatment. In particular, a TMS treatment method begins TMS treatment by applying an initial set of parameters for magnet orientation, power and frequency, and during the course of treatment one or more of these parameters is modified by patient feedback based on the acute experience of the patient during the TMS treatment. Systems for such intra-session control of TMS treatment may include one or more patient inputs, permitting feedback from the patient to modify the ongoing TMS treatment.

For example, described herein are methods including the steps of setting initial configuration parameters for TMS stimulation, stimulating the patient, and receiving direct feedback from the patient based on the acute response of the patient to the TMS treatment, and modifying the TMS treatment based on the feedback. The feedback received from the patient may be control feedback. For example, the patient may manipulate a control or other input for adjusting one or more of the parameters directly. Thus, the patient may tune or adjust a parameter based the patient's experience of one or more acute effects of the TMS therapy.

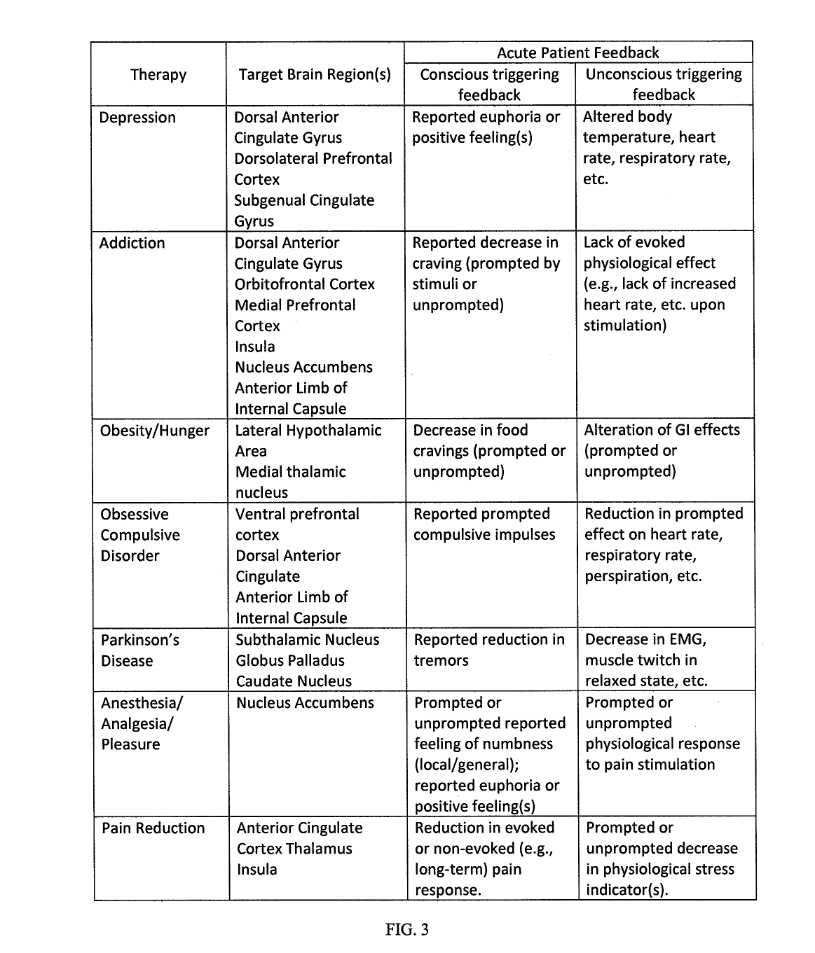

An acute experience of the effect of TMS therapy may be effect that is directly or indirectly associated with the disorder, behavior or condition being treated. For example, FIG. 3 illustrates different therapies that may be treated using TMS, and particularly deep-brain TMS. TMS therapies such as those described may have acute effects that are consciously experienced by the patient, as well as acute effects that are not consciously experienced. As indicated by the last two columns, these conscious and unconscious acute effects may be used as feedback, e.g., triggering feedback, to modify the applied TMS therapy. In particular, the patient may be allowed to adjust a TMS parameter based on the conscious experience of the TMS therapy. For example, a patient being treated for depression may manipulate the position, intensity or frequency of stimulation during the treatment until an acute effect such as a release from the depression or an experience of euphoria is experienced. In some variations the unconscious acute effects of the TMS stimulation may also (or alternatively) be used to adjust one or more parameters of TMS stimulation. In other variations, an unconscious effect of TMS stimulation must be present in order for the system to allow the patient to consciously modify a TMS parameter during treatment. FIG. 3 illustrates various conscious and unconscious effects that may be used to trigger feedback. These examples are not exhaustive, and other effects may be used. Effects that are directly or indirectly correlated with the therapy being applied are of particular interest.

When the triggering feedback is conscious, an alert patient typically manipulates a control to alter one or more stimulation parameter. In practice, the control manipulated may be a handheld control (e.g., button, mouse, joystick, touch screen, etc.), and may be configured so that a patient may manipulate it without moving his or her head or otherwise disturbing the arrangement of the TMS system to the patient's head.

Unconscious triggering feedback may be input from one or more sensors that feed information to the TMS system, including a controller. Thus, monitoring physiological information may be fed back into a controller that adjusts stimulation parameters after analyzing the physiological information. The triggering feedback may be an induced stimulation effect (e.g., identifying an increase or decrease in heart rate, blood pressure, etc.). As used herein, an unconscious triggering feedback measured from the patient is an acute effect that is downstream of the direct effect of the magnetic field applied to the brain region. Thus, the unconscious trigger feedback is not merely an imaging of the brain region being stimulated, showing the effect of TMS on the brain region targeted. Instead, the unconscious trigger feedback results from activation of one or more neural pathways downstream of the stimulated brain region.

A triggering feedback can be triggered as a respond to an inducing stimulus during TMS. For example, during TMS, the patient may be exposed to a stimulus configured to evoke a response that may be modulated by the TMS therapy. The modulation of an acutely evoked response to stimulus may be used to guide feedback for modifying one or more TMS parameters. For example, when treating a disorder such as obesity/overeating, the patient may be exposed to a visual stimulation (e.g., a picture of food) during the TMS therapy. The acute response to this stimulation may be an experience of cravings or an increase in heart rate, etc. The patient may adjust one or more parameters of the TMS therapy until a lessening of this acute response is experienced.

In some variations, the patient triggering feedback is a surrogate experience or an indirect experience, rather than a direct experience. For example, the experience may be an experience/perception that does not directly correlate with the therapy being treated. For example, an experience may be triggered by stimulation of a region of the brain that is nearby (e.g., superficial or adjacent) the target region.

The initial parameters may be set based on a best approximation of the therapeutic target and stimulation protocol. For example, the magnet (or magnets) may be a deep-brain target (e.g., see FIG. 3), and the initial parameters may include a magnetic field intensity that is based on the power applied to TMS electromagnet to stimulate the target without stimulating non-target regions. The frequency of stimulation may also be selected to stimulate (or inhibit) the target. In some variations, the starting parameters may be determined to be within a range of parameters that are calculated to be safe and potentially effective for the target region. This range of values for the parameters may serve as limits to the patient-controlled feedback/inputs.

For example, the initial parameters may include parameters for magnet location and/or orientation, strength of the applied magnetic field, pulse rate, and any other parameters applicable to access the target of interest based on available knowledge.

After receiving patient feedback, one or more parameters may be adjusted by or based on the feedback. As mentioned, the patient in some variations may consciously modify one or more parameters to increase/decrease an acute effect, preferably an effect correlated with the therapy. As part of the therapy or method of performing the therapy, the patient may be instructed on how to adjust/control the TMS stimulation based on a treatment effect. For example, the patient may be told to expect a particular acute effect, and how to modify the therapy based on the acute effect.

After modifying the one or more parameters based on the acute effect, the patient is again (or continues to be) stimulated and allowed to provide additional feedback. In this way a therapeutic response may be optimized. For example, the patient may be treated for acute pain, and during TMS treatment, may modify one or more parameters if the acute pain has not decreased. Feedback inputs may be repeated allowing continuous adjustments to aim, pulse rate, and other parameters. In some variations, a delay or pause may be experienced between the TMS application and the feedback input. Once the optimal effect has been achieved, the values of the parameters may be recorded for use in subsequent sessions. This may help formulate a treatment plan for that patient for that condition. Given the wide range of neurological conditions that are treatable using deep brain TMS, the patient may be potentially treated for multiple conditions that will require multiple configurations, not all of which will have a component of immediate feedback. It is understood that if the patient were being treated for an acute self-limited condition such as acute pain in conjunction with a dental procedure that subsequent treatment sessions may not be required. Alternatively, these optimized conditions may be used as initial parameters that may be later refined, since ‘drift’ of these parameters may be expected.

For any of the methods and devices described herein, suitable magnetic fields can be the type generated by TMS electromagnets such as the double-coil electromagnets available from Magstim, Ltd. (Wales, UK) or those generated by any other type of electromagnet used for TMS combined with pulse-generation systems such as the Rapid², also available from Magstim.

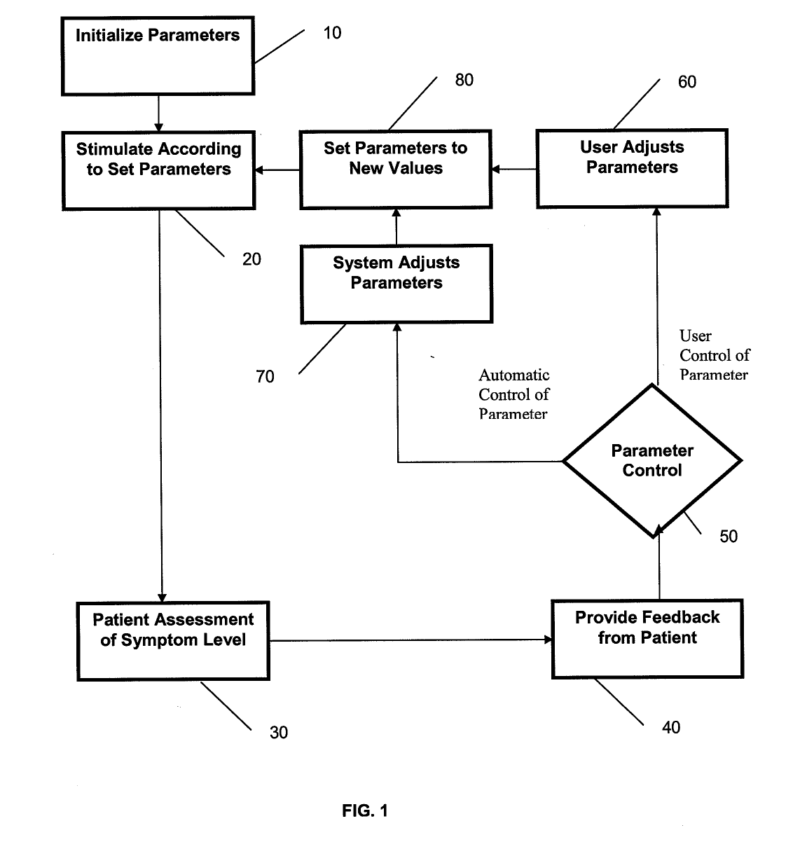

The flow chart FIG. 1 illustrates one example of TMS treatment method including intra-session feedback from the patient. The starting step 10 initializes the parameters. During the next step 20, the electromagnet or electromagnets are fired according to the initial set of parameters. Step 20 is the first step that may be continually in the loop including steps 20 through 80. The patient assesses the symptom level (for example level of pain) in step 30 and provides feedback in step 40. Step 40 can involve either a verbal report from the patient or direct patient input in a way (e.g., a Graphic User Interface on a computer) that can be processed automatically. If the parameter control 50 invokes user parameter control, then the user (physician, nurse, or technician) adjusts parameters in step 60. If the parameter control 50 invokes automatic parameter control according to incorporated algorithms, then the system adjusts parameters in step 70. Whether parameter adjustment occurs in step 60 or step 70, the new values are set in step 80 and the stimulation according to the newly set parameters occurs in step 20. The loop then continues until the session is completed. The process is applicable irrespective of the type of electromagnet(s) used, whether the electromagnet(s) are moved or not, the type of pulsing, mechanism to vary strength, setting of position or any other parameter.

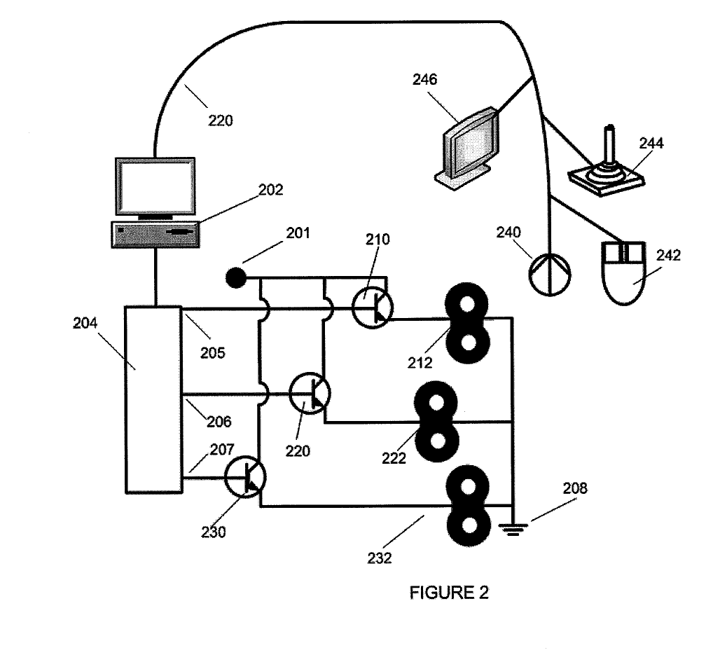

FIG. 2 illustrates one variation of a patient-configurable and optionally self-configuring system, including a control circuit. With this circuit, power is selectively applied to specific coils the array, at specific positions and pulse parameter. Computer 202 oversees the performance of multi-channel driver 204, ensuring that pulses are delivered at the right time, and to the proper coils. Multi-channel driver 204 controls TMS coil 212 via channel control line 205, and power transistor 210. Likewise, TMS coil 222 is controlled via channel control line 206 using power transistor 220, and TMS coil 232 is controlled via channel control line 207 using power transistor 230. The circuits to TMS coils 212, 222, and 232 are completed through ground connection 208. When power transistors (210, 220, 230) are activated by a corresponding control signal, they activate the corresponding coils by permitting passage of high voltages and currents from the capacitor bank power 201. In this manner, individual coil circuits may be switched on or off. The coil-activation time can also be controlled by supplying different frequencies of control pulses. Coils may also be moved between physical locations, under the guidance of computer 202 in accordance with the apparatus described in U.S. patent application Ser. No. 11/429,504 and No. 10/821,807.

Various controls may be used to provide feedback 220 to computer 202 regarding which parameters (e.g., coil positions, etc.) and how the parameters should be modified. Such controls may include, for example, transducer 240, mouse 242, joystick 244, or touch-screen computer 246. In the case of optimization of a treatment for Parkinson's disease, for example, an empiric testing procedure may be conducted with a transducer 240 in the form of an accelerometer or other motion sensor held in the patient's hand. The patient may then be asked to engage in specific tasks, such as attempting to remain still. Meanwhile, a signal processor examines the signal from the accelerometer, and determines how much tremor is associated with each task, as well as and how accurate and rapid the assigned movements are. During this process, a wide range of candidate stimulus parameter configurations, including position, intensity, and rate for one or more coils may be tested, either by automated or manual empirical processes. The optimal stimulus configuration can be determined empirically, for example, using a hierarchical algorithm to identify the optimal light position configuration for the specific patient. This optimization process can be carried out in an ongoing fashion, by monitoring over a period of days as the patient engages in their normal activities. The optimization process can thus gradually determine the best stimulus profile for the particular patient. At its extremes, all possible parameter configurations of all channels may be automatically tested over a period of time. In a more complex approach, rule-based, or artificial intelligence algorithms may be used to determine optimal parameters for each of the channels.

In the form of an accelerometer, transducer 240 also provides appropriate feedback when the coil is to minimize the amount of motor stimulation that occurs in the context of treatment with the system. By such an approach, the system may learn which positions achieve therapeutic goals without provoking untoward motor movement. One common side effect of rTMS treatment is inadvertent stimulation of the motor cortex, and consequently unintended elicitation of physical movement in the body of the patient. While motor cortex stimulation cannot always be avoided, it is prudent to avoid this phenomenon where possible, and in a manner that does not interfere with the overall treatment plan. For this purpose, inadvertent movement, as signaled by a transducer, may constitute feedback in the context of the present invention.

Various other input and testing procedures can be used depending upon the specific problem being treated. The patient's preference may be entered into a computer via text, graphic user interface, and/or device such as a mouse, track pad, trackball or joystick, or 3D optical tracking device. Various other brain-machine interfaces may also be used as part of the testing and optimization routine. It will be appreciated that the optimization process may be conducted in an open-loop (manual device configuration) or closed loop (fully automated device configuration) manner.

If appropriate measures of patient performance (for example freedom from tremor as measured by an accelerometer) are detected, this information can be automatically fed back to computer 302 for storage in a database. Computer 302 can use the stored information in accordance with algorithms and artificial intelligence methods to determine a suitable stimulation solution using driver 204.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention, which is set forth in the following claims.

### REFERENCES

Avery, D. H., Holtzheimer III, P. E., Fawaz, W., Russo, Joan, Neumaier, J. and Dunner, D. L., Haynor, D. R., Claypoole, K. H., Wajdik, C. and P. Roy-Byrne, “A Controlled Study of Repetitive Transcranial Magnetic Stimulation in Medication-Resistant Major Depression,” Biological Psychiatry, 2005;59:187-194.
Bohning, D. and M. George, “Method, apparatus, and system for automatically positioning a probe or sensor,” U.S. patent application Ser. No. 10/991,129.
Fox, P. and J. Lancaster, “Apparatus and Methods for Delivery of Transcranial Magnetic Stimulation,” U.S. Pat. No. 7,087,008.
George, M. S., Kozel, F. A., and D. E. Bohning, “Functional Magnetic Resonance Imaging Guided Transcranial Magnetic Stimulation Deception Inhibitor,” U.S. patent application Ser. No. 10/521,373.
Ives, J. R., Pascual-Leone, A. and Q. Chen, “Method and apparatus for monitoring a magnetic resonance image during transcranial magnetic stimulation,” U.S. Pat. No. 6,198,958.
Ives, J. R. and A. Pascual-Leone, “Method and apparatus for recording an electroencephalogram during transcranial magnetic stimulation,” U.S. Pat. Nos. 6,266,556 and 6,571,123.
Mayberg, H. S., Lazano, A. M., Voon, V., McNeely, H. E., Seminowicz, D., Hamani, C., Schwalb, J. M., and S. H. Kennedy, “Deep brain stimulation for treatment-resistant depression. Neuron,” 2005 Mar. 3; 45:651-60.
Mishelevich, D. J. and M. B. Schneider, “Trajectory-Based Transcranial Magnetic Stimulation,” U.S. patent application Ser. No. 11/429,504.
Riehl, M. E., “Determining stimulation levels for transcranial magnetic stimulation,” U.S. Pat. No. 7,104,947.
Schneider, M. B. and D. J. Mishelevich, “Robotic apparatus for targeting and producing deep, focused transcranial magnetic stimulation,” U.S. patent application Ser. No. 10/821,807.
Tanner, P., “Methods and Devices for Transcranial Magnetic Stimulation and Cortical Cartography,” U.S. Pat. Nos. 6,830,544 and 7,239,910.
Tanner, P., Hartlep, A., Wist, H., Wendicke, K. and T. Weyh, “Method and Device for Transcranial Magnetic Stimulation,” U.S. Pat. No. 7,008,370.

What is claimed is:

1. A patient-configurable Transcranial Magnetic Stimulation (TMS) method that allows a patient to dynamically modify the TMS while a TMS procedure is being performed, the method comprising:
applying Transcranial Magnetic Stimulation to a first site in the patient's brain, at a first magnetic field intensity and a first stimulation frequency;
changing one or more of the site, intensity or the frequency of the TMS stimulation based on input from the patient, wherein the patient changes one or more of the site, intensity or frequency of the TMS stimulation based on the patient's experience of the applied TMS stimulation; and
applying Transcranial Magnetic Stimulation to the patient at the new site, intensity or frequency of TMS stimulation.

2. The method of claim 1, further comprising providing a stimulus to prompt a patient experience that is modified during the TMS procedure.

3. The method of claim 2, wherein the stimulus comprises a visual stimulus.

4. The method of claim 2, wherein the stimulus comprises a tactile stimulus.

5. The method of claim 1, wherein the step of changing one or more of the site, intensity or the frequency of the TMS stimulation comprises allowing the patient to manipulate a handheld control to alter one or more of the site, intensity or frequency of the TMS stimulation.

6. The method of claim 5, wherein the patient may alter the site, intensity or frequency of the TMS stimulation only within a predetermined range for each of the site, intensity or frequency.

7. The method of claim 1, wherein the step of changing one or more of the site, intensity or the frequency of the TMS stimulation is performed while applying Transcranial Magnetic Stimulation to the patient.

8. A patient-configurable Transcranial Magnetic Stimulation (TMS) method that allows a patient to dynamically modify the TMS while a TMS procedure is being performed, the method comprising:
positioning a plurality of TMS electromagnets to apply electromagnetic energy to a deep brain target site;
applying TMS to the target site at a magnetic field intensity and a stimulation frequency;
enabling the patient to change one or more of the position of the TMS electromagnet, the intensity of the TMS stimulation, or the frequency of the TMS stimulation based the patient's experience of the applied TMS stimulation; and
applying Transcranial Magnetic Stimulation to the patient at the changed position of the TMS electromagnet, intensity of the TMS stimulation, or frequency of TMS stimulation.

9. The method of claim 8, further comprising providing a stimulus to prompt a patient experience that is modified during the TMS procedure.

10. The method of claim 9, wherein the stimulus comprises a visual stimulus.

11. The method of claim 9, wherein the stimulus comprises a tactile stimulus.

12. The method of claim 8, wherein the step of enabling the patient to change one or more of the position of the TMS electromagnet, the intensity of the TMS stimulation, or the frequency of the TMS stimulation comprises allowing the patient to manipulate a handheld control.

13. A system for applying Transcranial Magnetic Stimulation (TMS), the system comprising:
at least one TMS electromagnet configured to apply TMS to a site in a patient's brain;
a controller configured to control the TMS electromagnet to apply TMS to the site in a patient's brain at a magnetic field intensity and a frequency of stimulation; and
a patient feedback input connected to the controller, configured to allow the patient to adjust one or more of the site of application of the TMS in the patient's brain, the magnetic field intensity of the applied TMS, or the frequency of the TMS stimulation during a TMS procedure on the patient.

14. The system of claim 13, wherein the at least one TMS electromagnet comprises a plurality of TMS electromagnets configured to be positioned to apply TMS to a site in a patient's brain at a magnetic field intensity and a frequency of stimulation.

15. The system of claim 13, wherein the controller is configured to coordinate the stimulation applied by a plurality of TMS electromagnets to apply TMS to a deep brain target.

16. The system of claim 13, wherein the patient feedback input comprises a joystick.

17. The system of claim 13, wherein the patient feedback input comprises a mouse.

18. The system of claim 13, wherein the controller is configured to limit the adjustment of the site of application of the TMS in the patient's brain, the magnetic field intensity of the applied TMS, and the frequency of the TMS stimulation by the patient feedback input so that these parameters remain within a predetermined range of values.

19. A system for applying Transcranial Magnetic Stimulation (TMS), the system comprising:
a plurality of TMS electromagnets configured to apply TMS to a deep brain target site in a patient's brain;
a controller configured to control the plurality of TMS electromagnets to apply TMS to the target site in the patient's brain at a magnetic field intensity and a frequency of stimulation; and
at least one patient feedback input configured to allow the patient to adjust one or more of the site of application of the TMS in the patient's brain, the magnetic field intensity of the applied TMS, or the frequency of the TMS stimulation during a TMS procedure on the patient.

20. The system of claim 19, wherein the patient feedback input is selected from the group consisting of a joystick, a mouse, a touch screen, and a motion sensor.
